(19) United States
(12) Patent Application Publication
(10) Pub. No.: US 2010/0161059 A1
(43) Pub. Date: Jun. 24, 2010

(54) INTERVERTEBRAL IMPLANT FOR THE HUMAN OR ANIMAL BODY

(75) Inventors: Philippe Vincent-Prestigiacomo, Lacanau-Ocean (FR); Nicolas Roth, Bordeaux (FR)

Correspondence Address: CANTOR COLBURN, LLP, 20 Church Street, 22nd Floor, Hartford, CT, 06103, US

(73) Assignee: CREASPINE, Pessac (FR)

(21) Appl. No.: 12/594,848

(22) PCT Filed: Mar. 14, 2008
(86) PCT No.: PCT/FR08/00335

§ 371 Date: Oct. 6, 2009

(30) Foreign Application Priority Data
Apr. 13, 2007 (FR) 0702680

(51) Int. Cl. A61F 2/44

(52) U.S. Cl. 623/17.16

(57) ABSTRACT

This intervertebral implant (17) for the human or animal body, suitable for an anterior or posterior location, comprising two relatively rigid pillars (19, 20) connected to each other only by at least two bearing portions (21, 22) able to interact with respective vertebral endplates (7) of the spine, at least one of said bearing portions (21, 22) comprising at least one relatively flexible zone allowing this bearing portion to at least partially conform to the shape of the associated vertebral endplate (7) when said implant (17) is positioned between said two endplates.

### TECHNICAL FIELD OF THE INVENTION

The present invention relates to the field of spinal surgery of the human or animal body, and more particularly to intervertebral implants.

### BRIEF SUMMARY OF RELATED ART

There are conventionally two categories of intervertebral implants:
the first category relates to implants called intersomatic implants, designed to replace the intervertebral disks, and therefore to be placed between two adjacent vertebrae;
the second category relates to implants of the VBR (Vertebral Body Replacement) type, designed to partly or completely replace one or more vertebrae.

All these implants, with which bone grafts are usually associated, make it possible to fuse the vertebral bodies with which they interact, and therefore to remedy various spine pathologies.

Shown schematically in FIG. 1 attached hereto is a first type of intervertebral implant 1 of the prior art.

This implant, which may be made of biocompatible plastic such as PEEK (polyether-etherketone) or of metal such as a titanium alloy, has a substantially parallelepipedal shape, and is furnished with orifices 3 allowing the installation of bone graft and the contact of this graft with the host bone.

FIG. 2 shows a vertebra 5 resting on this implant: as can be seen in this figure, the vertebral endplate 7 of this vertebra which interacts with this implant has a certain concavity.

Therefore, the vertebra 5 in reality rests only on the edges 8, 9 of the implant 1: this pressure in places causes very great stresses on the vertebral endplate 7 and in the adjacent vertebral body 11, which may in time cause a weakening or even a collapse of the vertebra 5.

This concentration of stresses on the edges of the implant is illustrated by the graph of FIG. 3.

In this graph, the abscissa corresponds to the curvilinear abscissa on the vertebral endplate measured in mm on the axis x of FIG. 2, and the ordinate indicates the pressure exerted on the vertebra 5 by the implant 1 expressed in Von Mises stress in MPa: the two stress peaks 12a, 12b that can be seen in this graph correspond to the bearing zones of the edges 8, 9 of the implant 1 on the vertebral endplate 7.

To alleviate this drawback, thought has been given, in the prior art, to manufacturing implants whose faces designed to come to bear against the vertebral endplates have a convexity matching the concavity of these endplates.

An example of such an implant 13 is shown in FIG. 4.

In practice, no vertebra has the same concavity: therefore, the implant of FIG. 4 will correctly match the shape of certain vertebrae, but will not be suitable for others.

In concrete terms, this means that for certain vertebrae, the user will find himself in the situation shown in FIG. 5, where contact between the implant 13 and the vertebral endplate 7 is in isolated places, causing a stress peak in the center of the vertebral endplate as can be seen in FIG. 6 at 15.

The user then finds himself again in an unfavorable configuration likely to lead to the weakening or even the collapse of the vertebra 5.

To remedy these drawbacks, it has been proposed, in documents U.S. 2004/267367, WO2004/064693 and WO2006/127849, to use intervertebral implants whose bearing portions capable of interacting with respective vertebral endplates of the spine, have a certain flexibility.

By virtue of this flexibility, it is possible to obtain a distribution of the bearing forces of the implant on each associated vertebral endplate, and thereby prevent the stress concentrations that are likely to damage the vertebra.

These intervertebral implants with flexible bearing zones of the prior art however have the drawback of not allowing optimal revascularization and bone fusion.

### BRIEF SUMMARY OF THE INVENTION

The invention provides an intervertebral implant for the human or animal body suitable for an anterior or posterior placement, comprising two relatively rigid pillars, connected together only by at least two bearing portions capable of interacting with respective vertebral endplates of the spine, at least one of said bearing portions comprising at least one relatively flexible zone allowing this bearing portion to at least partially match the shape of the associated vertebral endplate when said implant is placed between said two endplates.

By virtue of this structure of pillars connected together by bearing portions, the vertebral implant with flexible bearing zones according to the invention defines a cavity that favors bone fusion and revascularization. This cavity in particular allows the placement of bone grafts.

This particular structure distinguishes the implant according to the invention from those of the prior art, which are solid and therefore form a screen to bone fusion and revascularization.

The fact that the two pillars are connected together only by two bearing portions gives the implant a particularly simple structure.

Surprisingly, it was noted that providing a cavity in the implant was compatible with envisaging flexible bearing zones: with appropriate design, within the scope of those skilled in the art having the conventional rules of material strength, it was found that it was possible to reconcile flexibility and cavity without exposing the implant to a risk of being crushed by the adjacent vertebrae.

Until the present invention, it was thought that it was necessary for an implant with flexible bearing zones to be solid to withstand crushing.

According to other optional features of the implant according to the invention:
said bearing portions comprise at least two relatively flexible plates connecting said pillars together: this embodiment makes it possible to obtain the desired flexibility by configuring the bearing portions as plates, that is to say by working on the shape and thickness of the material forming the implant and therefore on its intrinsic elasticity;
said bearing portions comprise bearing zones secured to said pillars and a relatively rigid plate connected to said pillars by relatively flexible arms: this is a variant of the preceding embodiment, in which the desired flexibility is obtained by the means for connecting the plates to the body;
said body and said plates define an external volume and an internal cavity that are oblong: this particular configuration makes it possible to obtain an implant which is particularly well suited to the various concavities of the vertebral endplates of the vertebrae;
said plates are provided with vascularization orifices: these orifices make it possible to reconstitute the vascular networks in the zone of the implant;
said bearing portions comprise retention striae: these striae contribute to immobilizing the implant between the associated vertebral endplates;
this implant is formed in one block in a metal biocompatible material such as Ti₆Al₄V (titanium alloy) or a biocompatible polymer such as PEEK, PLLA or PGA: this embodiment in a block is particularly simple and can be obtained respectively by machining the metal alloy or by molding the polymers; these materials are very widely used in the field of surgical implants.

### BRIEF DESCRIPTION FO THE DRAWINGS

Other features and advantages of the present invention will appear in the light of the reading of the following description and on examination of the appended figures in which:

FIG. 1 is a view in perspective of a first implant of the prior art, described above,

FIG. 2 is a view of this implant interacting with a vertebra,

FIG. 3 is a graph representing the stresses transmitted by the implant to the vertebra,

FIGS. 4 to 6 are views similar to those of FIGS. 1 to 3 for a second implant of the prior art, described above,

FIG. 7 is a view in perspective of an implant according to the invention,

FIG. 8 is a view of this implant interacting with a vertebra, with no compression,

FIG. 9 is a view of this implant interacting with a vertebra subjected to a compression force,

FIG. 10 is a graph representing the stresses transmitted by the implant 17 to the vertebra 5 in a compression situation,

FIG. 11 is a view in perspective of a variant of the implant according to the invention, and

FIG. 12 is a front view of this variant.

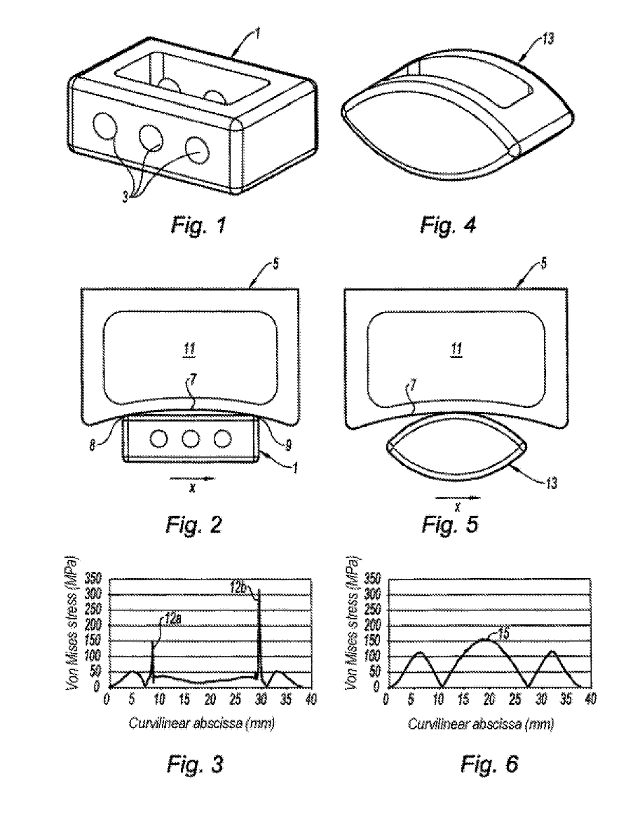

### DETAILED DESCRIPTION OF THE INVENTION

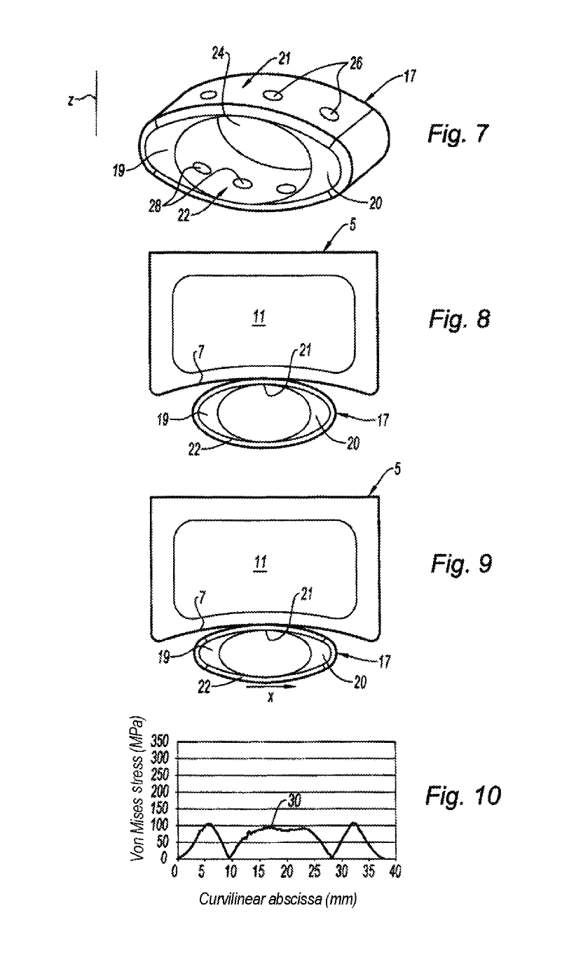

Reference is now made to FIG. 7, in which it can be seen that an implant according to the invention may comprise two pillars 19, 20 connected together by two plates 21, 22.

The two pillars 19, 20, which form the body of the implant 17, have a high resistance to vertical forces, that is to say to the forces oriented in the straight line Z of FIG. 7.

The plates 21, 22, for their part, form bearing portions offering a relative flexibility relative to the pillars 19, 20: the ratio of the elastic deformations, under a given load, between the rigid pillars and the flexible plates is typically of the order of 1 to 10.

Preferably, as shown, the pillars 19, 20 and the plates 21, 22 define an external volume on the one hand and an internal cavity 24 on the other hand with oblong and preferably elliptical sections with indistinguishable axes.

Also provided is a plurality of vascularization orifices 26, 28 on the plates 21, 22.

Provision may also be made for these plates 21, 22 to be furnished with retention striae (not shown) on their outer surfaces.

Advantageously, the implant 7 may be formed in a single block in a material such as PEEK (polyether-etherketone), PLLA (polylactic acid) or PGA (propyleneglycol alginate). In this case, the implant 17 may be obtained for example by molding or by machining.

Reference is now made to FIG. 8, which shows the implant 17 according to the invention in contact with the vertebral endplate 7 of a vertebra 5 before being placed under compression, that is to say before being subjected to the forces transmitted by the spine.

As can be seen in this figure, the type of contact between the implant 17 and the vertebral endplate 7 of the vertebra 5 is of the same type as that shown in FIG. 5: this contact is substantially in isolated places.

Reference is now made to FIG. 9, in which the implant 17 can be seen in a compression situation, that is to say when it is placed in the spine and subjected to the forces transmitted by the latter.

As can be seen in this figure, the two relatively flexible plates 21, 22 have deformed so as to come closer to one another, which makes it possible to considerably increase the surface of contact of these plates with the adjacent vertebral endplates.

In this way it is possible to obtain an optimal distribution of the stresses transmitted by the implant 17 on the vertebra 5 as illustrated by the flat zone 30 in the graph of FIG. 10.

The high stresses in isolated places likely to damage, or even ruin the vertebra 5 are removed in this way.

It will be noted that, despite this flexibility of the plates 21, 22, the implant 17 continues to retain its structural function by virtue of the relatively rigid pillars 19, 20.

It will also be noted that the relative flexibility of the plates 21 and 22 allows the implant 17 according to the invention to adapt to practically any degree of concavity of the vertebral endplates of the vertebrae.

Therefore, it is possible to considerably reduce the number of different implants adaptable to all the vertebrae.

The orifices 26, 28 formed in the relatively flexible plates 21, 22 allow a rapid reconstitution of the vascular network between the vertebrae.

The internal cavity 24 makes it possible to install a bone graft recommended for fusion or for arthrodesis.

It will also be noted that the relative flexibility of the plates 21 and 22 makes it possible to act upon the surfaces of the associated vertebral endplates, and therefore to promote regrowth of the bone, by virtue of Wolff's law, well known to those skilled in the art.

It will also be noted that the shape of the implant shown in FIGS. 7 to 9 is in no way limiting.

Therefore, while a symmetrical shape as shown in these figures is adapted to a placement of the anterior type (that is to say through the front of the body of the patient), an asymmetrical shape could be suitable for a posterior placement (that is to say via the back of the body of the patient).

Naturally, other variants of the implant according to the invention could be envisaged.

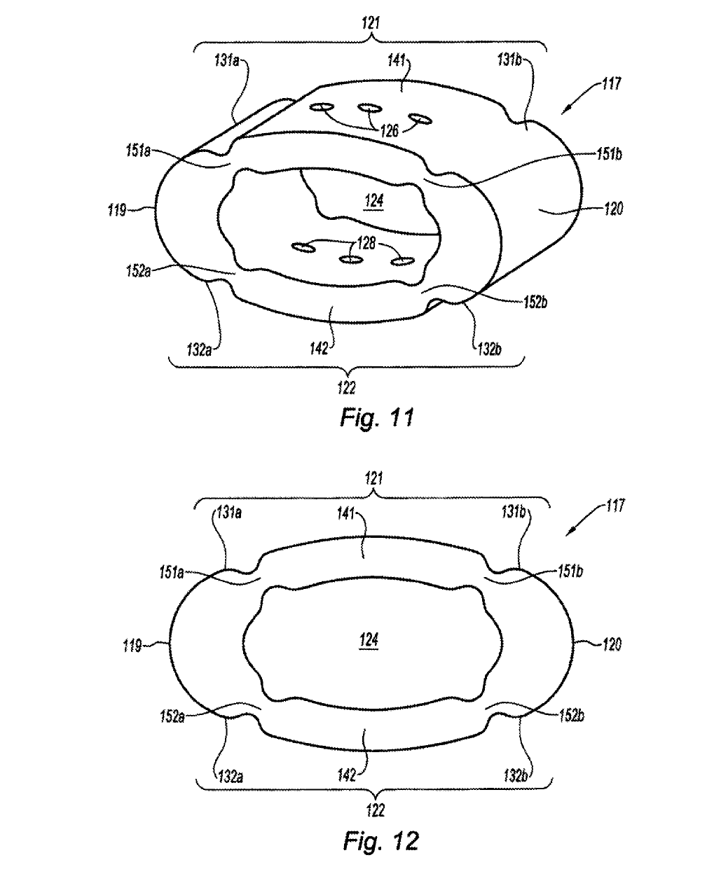

It is in this way, for example, that it is possible to envisage the implant variant 117 shown in FIGS. 11 and 12, and in which the portions bearing against the vertebral endplates are formed on the one hand by bearing zones 131a, 131b and 132a, 132b that are secured to the relatively rigid pillars 119, 120 forming the body of this implant and, on the other hand, by two relatively rigid plates 141, 142 connected to these pillars by relatively flexible arms 151a, 151b and 152a, 152b.

1. An intervertebral implant for the human or animal body, suitable for an anterior or posterior placement, comprising:
two relatively rigid pillars connected together only by at least two bearing portions capable of interacting with respective vertebral endplates of the spine,
at least one of said bearing portions comprising at least one relatively flexible zone allowing the bearing portion to at least partially match a shape of the associated vertebral endplate when said implant is placed between said two endplates.

2. The implant as claimed in claim 1, wherein said bearing portions comprise at least two relatively flexible plates connecting said pillars together.

3. The implant as claimed in claim 1, wherein said bearing portions each comprise bearing zones that are secured to said pillars and a relatively rigid plate connected to said pillars by relatively flexible arms.

4. The implant as claimed in claim 2, wherein said body and said plates define an external volume and an internal cavity that are oblong.

5. The implant as claimed in any one of claim 2, wherein said plates are provided with vascularization orifices.

6. The implant as claimed in claim 1, wherein said bearing portions comprise retention striae.

7. The implant as claimed in claim 1, wherein it is formed in one block in a metal biocompatible material.

8. The implant as claimed in claim 7, wherein the biocompatible material comprises Ti₆Al₄V.

9. The implant as claimed in claim 7, wherein the biocompatible material comprises a biocompatible polymer comprising at least one of PEEK, PLLA and PGA
